(19) United States
(12) Patent Application Publication
(10) Pub. No.: US 2020/0390711 A1
(43) Pub. Date: Dec. 17, 2020

(54) CUSTOMIZABLE CANNABINOID POWDER AND PRODUCTION METHODS

(71) Applicant: PHYTOCANNETICS INC., Carlsbad, CA (US)

(72) Inventors: Stephanie Tappan Schaefer, Carlsbad, CA (US); John Chrysostom Miller, Carlsbad, CA (US)

(21) Appl. No.: 16/852,076

(22) Filed: Apr. 17, 2020

Related U.S. Application Data
(60) Provisional application No. 62/835,957, filed on Apr. 18, 2019.

(51) Int. Cl. A61K 9/19; A61K 31/352; A61K 31/05; A61K 36/185; A61K 9/08

(52) U.S. Cl. CPC A61K 9/19; A61K 31/352; A61K 9/08; A61K 36/185; A61K 31/05

(57) ABSTRACT

Methods of producing a phytocannabinoid powder having a customizable cannabinoid content are disclosed. The phytocannabinoid powder comprises the whole of cannabis flowers and leaves and is self-emulsifying when mixed into water or other aqueous solution. The phytocannabinoid powder, and its constituents, may be used in food, nutritional supplement, and beverage products for humans, as well as in feed and supplements for pets and livestock.

### CROSS-REFERENCE TO RELATED APPLICATIONS

This application claims priority under 35 U.S.C. § 119(e) to U.S. Provisional Application No. 62/835,957, filed Apr. 18, 2019.

### BACKGROUND

Conventional large-scale cannabis harvesting methods involve cutting the cannabis plant at the stem and hanging it upside down to allow the plant material to dry over the course of days or weeks. Once the flowers are at the desired dryness, they are generally cured by storing at controlled conditions over the course of days or weeks to allow for potency and flavor to develop before the flowers are smoked or extracted to isolate cannabinoids within the plant matter.

As the medical aspects of cannabis and cannabinoids are gaining greater appreciation and use, there is an increasing demand for methods of consumption that do not require smoking. Cannabinoid beverages are one such method, but they frequently suffer from the difficulty of getting and keeping the oily cannabinoids in the aqueous solution that forms most beverages, having to rely upon emulsifiers that may reduce the shelf-stability, shelf-life and taste of the beverage.

### SUMMARY

In some embodiments, a method of producing a customizable phytocannabinoid powder is described. In some embodiments, the method comprises cutting at least some of the stalks and branches of a cannabis plant to remove flowering buds and leafy materials showing signs of resin production. The method further comprises stripping the flowering buds and leafy materials from the stalk and branches cut from the cannabis plant, such that the flowering buds and leafy materials form a biomass. The method further comprises pressing the biomass, such that the biomass separates into a liquid content and a solid content. The method further comprises making the liquid content into a freeze-dry powder, comprising freeze-drying the liquid content from pressing and optionally processing the freeze-dried liquid content such as by grinding or sifting to form a freeze-dry powder. The juice or liquid and the freeze-dry powder made therefrom contain a blend of cannabinoids that is different than what results when the biomass is allowed to age, dry and/or cure before pressing. The method further comprises making the solid content into a ground flower powder, comprising treating the solid content such as by drying, grinding and/or sonicating. The method further comprises adding the ground flower powder to the freeze-dry powder, such that the ground flower powder and the freeze-dry powder become emulsified and form the customizable phytocannabinoid powder or “phytopowder.”

The freeze-dry powder, ground flower powder and phytocannabinoid powder all have the ability to self-emulsify when combined with water or other aqueous materials or solutions, making them a better way of incorporating cannabinoids into beverages or beverage concentrates or mixes than standard hash oils.

In some embodiments, the solids that become the ground flower powder are extracted to remove cannabinoids, such as by CO₂, butane, ethanol or other solvent, either before or after drying and/or grinding/sonicating. The chemically extracted cannabinoids may be further optionally purified and/or isolated such as by HPLC or CPC or other method.

In some embodiments, some or all of the cannabinoids extracted from the solid materials are mixed back into the phytocannabinoid composition, freeze-dry powder and/or ground flower powder. In some embodiments, cannabinoids from other sources are mixed into the phytocannabinoid composition, freeze-dry powder and/or ground flower powder. The cannabinoids mixed into these materials may be all or some of the cannabinoids from the extraction or other source, so as to create a custom cannabinoid profile for the material.

### BRIEF DESCRIPTION OF THE DRAWINGS

FIG. 1 is a flow diagram illustrating a method of making a customizable phytocannabinoid powder.

FIG. 2 is a flow diagram illustrating a method of making a freeze-dry powder.

FIG. 3 is a flow diagram illustrating a method of making a ground flower powder.

### DETAILED DESCRIPTION

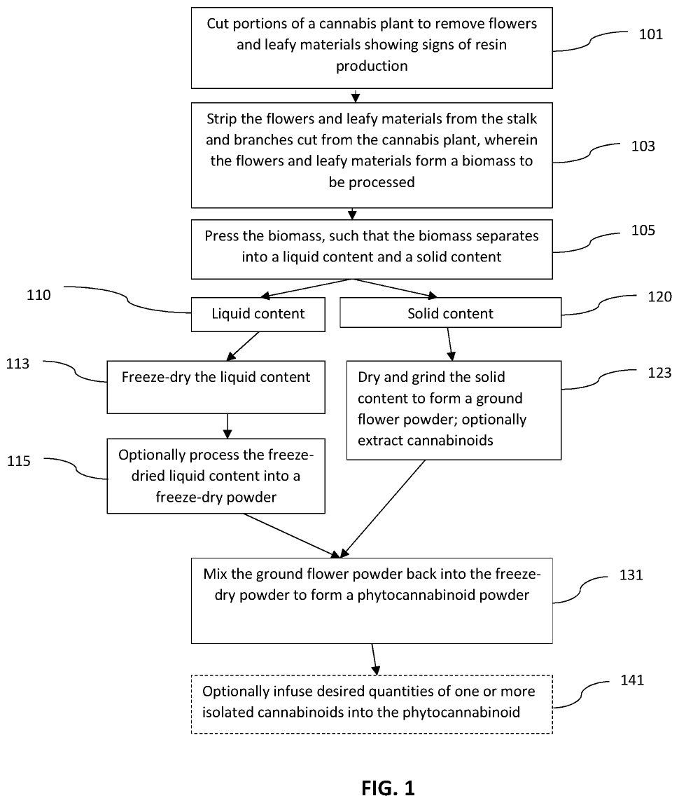

FIG. 1 is a flow diagram illustrating one example of a method of producing a customizable phytocannabinoid powder. The method comprises cutting at least some of the stalks and branches of a cannabis plant to remove flowers and leafy materials showing signs of resin production 101. The method further comprises stripping the flowers and leafy materials from the stalk and branches cut from the cannabis plant 103, such that the flowers and leafy materials form a biomass. The method further comprises pressing the biomass 105, such that the biomass separates into a liquid content 110 and a solid content 120. The method further comprises making the liquid content 110 into a freeze-dry powder, comprising lyophilizing or freeze-drying the liquid content 113 and optionally processing the freeze-dried liquid content such as by grinding or sifting into a freeze-dry powder 115. The method further comprises making the solid content 120 into a ground flower powder 123 by treating the solid content including by drying and/or grinding or sonicating. The method further comprises adding the ground flower powder to the freeze-dry powder 131, such that the ground flower powder and the freeze-dry powder form the phytocannabinoid powder. The method may further comprise optionally infusing desired quantities of one or more of cannabinoids into the phytocannabinoid powder 141 to create a phytocannabinoid powder having a customized cannabinoid profile. When the phytocannabinoid powder is added to water or other aqueous solution or material, it is self-emulsifying and no additional or extraneous emulsifiers need to be added to keep the phytocannabinoid powder and its components in solution.

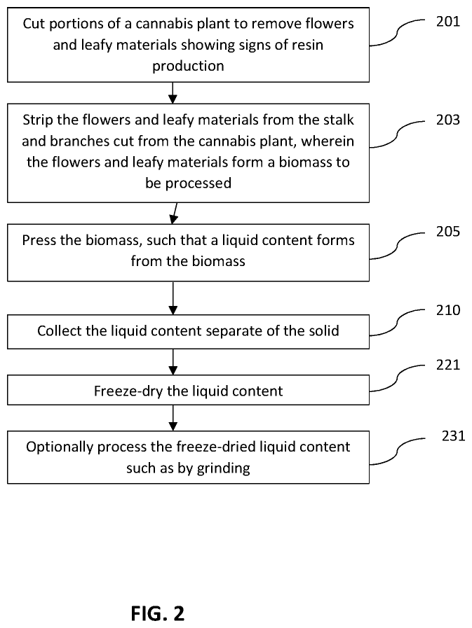

FIG. 2 is a flow diagram illustrating one example of a method of making a freeze-dry powder. The method comprises cutting at least some of the stalks and branches of a cannabis plant to remove flowers and leafy materials showing signs of resin production 201. The method further comprises stripping the flowers and leafy materials from the stalk and branches cut from the cannabis plant 203, such that the flowers and leafy materials form a biomass. The method further comprises pressing the biomass 205 to form a liquid content 210 that is separated from the solids and freeze-drying the liquid content 221. The method optionally comprises processing the freeze-dried liquid content such as by grinding or sifting 231.

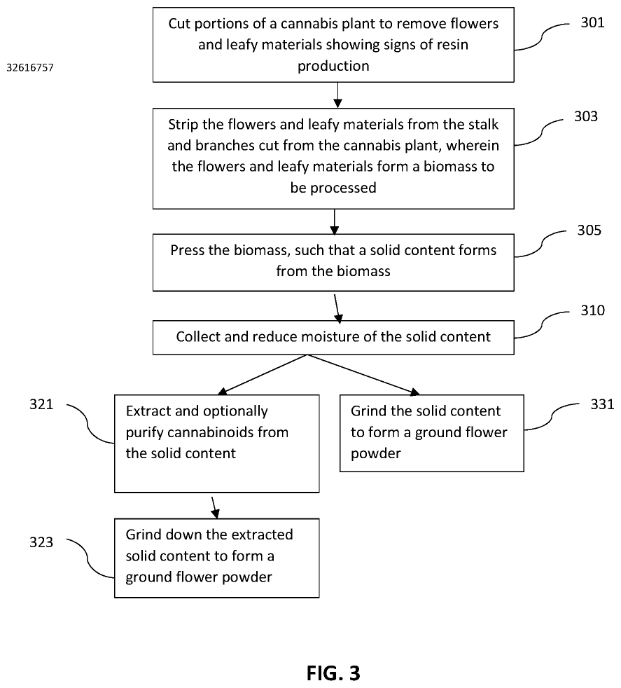

FIG. 3 is a flow diagram illustrating one example of a method of making a ground flower powder. The method comprises cutting at least some of the stalks and branches of a cannabis plant to remove flowers and leafy materials showing signs of resin production 301. The method further comprises stripping the flowers and leafy materials from the stalk and branches of the cannabis plant 303, such that the flowers and leafy materials form a biomass. The method further comprises pressing the biomass 305 to form a solid content 310 that is separated from the juice or liquid and dried to reduce its moisture content. The method optionally further comprises extracting cannabinoids from the dried solids 310 using solvent extraction methods and further optionally purifying the extracts 321, followed by grinding down the extracted isolated cannabinoids to form a ground flower powder 323. Alternatively, the method comprises grinding down the solid content 310 to form the ground flower powder 331.

In some embodiments, the solid content, ground flower powder, liquid content, freeze-dry powder, and/or phytocannabinoid powder may be treated by mixing or infusing them with isolated cannabinoids, either individual cannabinoids or mixtures thereof, to create a customized cannabinoid profile for the material. The cannabinoid content can be customized by adding cannabinoids from any source, including those isolated from the solid component following pressing.

Cannabinoids used to create a customized cannabinoid profile include, but are not limited to, cannabigerolic acid (CBG-A), cannabigerol (CBG), tetrahydrocannabinolic acid (THC-A), tetrahydrocannabinol-C4 (THC-C4), tetrahydrocannabivarinic acid (THCV-A), delta-9-tetrahydrocannabinol (Δ9THC), delta-8-tetrahydrocannabinol (Δ8THC), tetrahydrocannabivarin (THCV), cannabinolic acid (CBN-A), cannabinol (CBN), cannabidiolic acid (CBD-A), cannabidivaric acid (CBDV-A), cannabidiol (CBD), cannabidivarin (CBDV), cannabichroic acid (CBC-A), cannabichromene (CBC), cannabicyclol acid (CBC-A).

The methods summarized in the above FIGS. 1, 2, and 3 are only examples of methods. Variations and details of the methods, materials, and compositions include the following.

### Cannabis Plants

The cannabis plant used in the methods and compositions described herein can include, but are not limited to, Cannabis sativa, Cannabis indica, Cannabis ruderalis, or hybrid plants.

It is contemplated that the cannabis plants as described herein can be grown in soil, hydroponics, or aeroponics, and may be grown outdoors, indoors, under natural light, and/or under artificial light. The cannabis may be grown organically, without artificial fertilizers or pesticides.

### Continuous Harvesting

The portions of the cannabis plant used in the methods and materials described herein may include flowers, also known as buds, and leafy materials. These materials may be harvested from a plant by cutting the stalks and/or branches having leaves and/or flowers that appear ready for harvest such as by showing signs of resin production. Cutting of the stalks or branches may be done while leaving the remainder of the plant to grow and/or mature such that additional materials may be harvested at a later time. This is referred to herein as continuous harvesting.

Continuous harvesting of the cannabis plant may occur at the first sign of resin production, and may occur starting from about week 3 through about week 13 of the growth cycle of the cannabis plant, extending beyond the period of seed ripeness, depending upon factors such as growing conditions or the strain of cannabis being grown. The start of the growth cycle for a plant may be considered to be the beginning of flowering or when the first pistils form at any location on that plant.

The term “first sign of resin production” for a cannabis plant refers to when trichomes begin to appear on flowers or other portions of the plant. The type of chemicals, including cannabinoids and terpenes, created by the plant, and thus appearing within the juice of harvested plant matter, will change throughout the growing process. Accordingly, the juice will have a different constitution depending upon how long the plant has been flowering and how long the plant material is allowed to sit before juicing. Harvest will generally not occur until there are visible signs of resin production.

Once removed from the plant, the flowers and other leafy materials are stripped away from the stalks, branches and stems such as by trimming using scissors or a blade. The stalks, branches and stems generally have a much lower cannabinoid content and a woodier texture that make them less desirable for use in further process steps, although they may be used in further steps in some embodiments.

The cannabis flowers and leafy materials that are harvested should be pressed or flash frozen immediately, preferably within 3 hours, including two hours and one hour, of harvesting. Without being limited by theory, it is contemplated that flash-freezing plant materials within about 3 hours of detaching the plant materials from the living plant preserves a significant portion of the cells and living materials within the detached portion of the plant material. As such, the biomass formed from harvested cannabis flowers and leafy materials can be flash frozen immediately within 3 hours of harvesting at a temperature of below −30° F., including below −70° F., −60° F., −50° F., −40° F. or −30° F. The frozen biomass may then be pressed, either while frozen or after thawing, provided that the thawed biomass is pressed within about 3 hours of thawing.

### Pressing/Juicing

In some embodiments, the biomass harvested from the plant can be pressed or juiced immediately after the biomass is cut. In some embodiments, flash-frozen biomass is pressed or juiced before, during, or immediately after thawing. The pressing or juicing can be done on any equipment suitable for mechanically extracting, pressing or forcing out a juice, oil or liquid from solid plant matter, including but not limited to an extruder, a press (pneumatic, hydraulic, or screw), a blender, or a juicer.

The pressing creates a liquid portion and a solid portion that may be separated from each other by any suitable method including, but not limited to, filtering and decanting.

### Processing of the Liquid Portion

In some embodiments, the liquid content from the pressing of the biomass may be freeze-dried and optionally processed to form the freeze-dry powder. Freeze-drying or lyophilization removes water via sublimation from a product placed under a vacuum or reduced pressure at a temperature sufficiently low to cause water to freeze. In some embodiments, following freeze-drying the liquid content (i.e. the powder resulting from freeze-drying or lyophilization) is processed into the freeze-dry powder. Such processing may include, but is not limited to, grinding, screening, sifting and sonicating. For example, a material may be sifted or screened through one or more sizes of mesh to create a desired particle size range. In other embodiments, the lyophilized material is used without further processing.

The freeze-dry powder comprises aqueous and oily components separated from the bulk solid plant material, and includes cannabinoids, terpenes, alkaloids, chlorophyll and vitamins.

In some embodiments, the method further comprises flash-freezing the liquid content after pressing the biomass. Freezing may be done at a temperature of below about 0° F. For example, the temperature can be below −10° F., −20° F., −30° F., −40° F. or −50° F. The frozen liquid content may later be subject to additional processing, including lyophilization (i.e. freeze-drying).

In some embodiments, the juice from pressing may be consumed directly fresh or frozen (or previously frozen) by a human or other mammal or animal. Such juice may be consumed alone or in combination with other juices such as from fruits, vegetables, and other whole plants or plant matter, and/or other ingredients.

### Processing of the Solid Portion

In some embodiments, the solid content from the pressing of the biomass can be ground into the ground flower powder. The solid content may be dried to about 5% to 15% moisture, including 7%, 10%, and 12% moisture through methods that include, but are not limited to, dehydration, solar drying, hot air drying, bin drying, rotary drying, fluidized-bed drying, heated-surface contact drying, and freeze-drying. The dried material may then be ground by one or more methods to produce the ground flower powder, such methods including, but not limited to, milling, tumbling, sonicating, use of a blender or food processer or similar equipment, and the like. Sonication, treatment with high frequency sound or ultrasonic waves that may cause cavitation, may be preferred in some instances to create smaller and perhaps more uniform particle sizes as compared to materials that have not been sonicated.

In some embodiments, cannabinoids can be extracted from the solid content, by solvent extraction, including supercritical fluid extraction, using CO₂, butane, ethanol or other solvent. Other extraction methods may also be used. Extraction may occur before or after drying, and/or before or after grinding or sonication. Cannabinoids and other compounds such as terpenes, that are removed by chemical extraction may be optionally purified and/or isolated via HPLC or CPC or other methods. In some embodiments the ground flower powder includes a combination of extracted and unextracted materials.

The ground flower powder comprises dried plant cells and cell components, and cannabinoids and terpenes to the extent that they have not been removed via extraction or the initial pressing.

### Making Phytocannabinoid Powder

In some embodiments, the freeze-dry powder and/or the ground flower powder may be used alone, whereas in others they are combined to make a phytocannabinoid powder. (“PhytoPowder”). In some embodiments, the ratio of the freeze-dry powder to the ground flower powder in the phytocannabinoid powder is between 0.5:1 and 100:1. For example, the ratio may be 0.5:1, 1:1, 2:1, 3:1, 5:1, 8:1, 10:1, 20:1, 30:1, 40:1, 50:1, 60:1, 70:1, 80:1, 90:1, or 100:1. In other embodiments, the ratio of ground flower powder to freeze-dry powder in the phytocannabinoid powder is between 0.5:1 and 100:1, including 0.5:1, 1:1, 2:1, 3:1, 5:1, 8:1, 10:1, 20:1, 30:1, 40:1, 50:1, 60:1, 70:1, 80:1, 90:1, and 100:1.

One especially useful property of both the ground flower powder and the phytocannabinoid powder is that they can act as a natural emulsification agent or emulsifier. This allows the powder and its constituent cannabinoids and other oily compounds to mix with water or other aqueous material without readily separating. In preferred embodiments, there is no significant phase separation for at least 12 hours, including at least 1 day, at least 3 days, at least 10 days, at least 2 weeks, at least 1 month, at least 6 months, and at least 1 year.

The phytocannabinoid powder or PhytoPowder therefore comprises dried plant cells and cell components, and cannabinoids and terpenes and other components of the ground flower powder together with the aqueous and oily components separated from the bulk solid plant material, including cannabinoids, terpenes, alkaloids, chlorophyll and vitamins, from the freeze-dry powder together. It may further comprise additional cannabinoids or other components. The PhytoPowder is a whole-plant composition that self-emulsifies to keep the oily components in solution when blended with water or an aqueous solution.

### Customization of Cannabinoid Content

The phytocannabinoid powder or its components or component precursors (e.g. solid content, ground flower powder, liquid content, freeze-dry powder), may be mixed or infused with isolated cannabinoids, either individual cannabinoids or mixtures thereof, to create a material having a customized cannabinoid profile. This may similarly be done with terpenes, alkaloids, and/or other components of cannabis or other useful plants. The cannabinoid content may be customized by adding some or all of the cannabinoids isolated from solid component following pressing (the precursor to the ground flower powder) and/or from one or more other sources. In some embodiments, the cannabis plants used to make phytocannabinoid powder and/or the source of isolated cannabinoids includes, but is not limited to, Cannabis sativa, Cannabis indica, Cannabis ruderalis and hybrids thereof. Combining of the cannabinoids and other materials may be done by simple mixing of the materials together, taking care to not heat the mixture so as to alter any of the cannabinoids or other compounds.

Cannabinoids used to create a customized cannabinoid profile include, but are not limited to, cannabigerolic acid (CBG-A), cannabigerol (CBG), tetrahydrocannabinolic acid (THC-A), tetrahydrocannabinol-C4 (THC-C4), tetrahydrocannabivarinic acid (THCV-A), delta-9-tetrahydrocannabinol (Δ9THC), delta-8-tetrahydrocannabinol (Δ8THC), tetrahydrocannabivarin (THCV), cannabinolic acid (CBN-A), cannabinol (CBN), cannabidiolic acid (CBD-A), cannabidivaric acid (CBDV-A), cannabidiol (CBD), cannabidivarin (CBDV), cannabichroic acid (CBC-A), cannabichromene (CBC), cannabicyclol acid (CBC-A).

In some embodiments, a desired quantity of cannabinoids added to a material such as the solid content, ground flower powder, liquid content, freeze-dry powder, and/or phytocannabinoid powder, can be between 0.01 grams and 15 grams of cannabinoid per 100 grams of material to which the cannabinoids are added. For example, a desired quantity can be 0.01, 0.1, 0.3, 0.5, 1, 1.5, 2, 5, 7, 10, 12, and 15 grams per 100 grams of material.

In some embodiments, a desired quantity of cannabinoids in a material, including both those cannabinoids in the material itself before enrichment and those added to the material, is between 0.01% and 50% by weight. For example, a desired quantity can be 0.01%, 0.1%, 0.3%, 0.5%, 1%, 1.5%, 2%, 5%, 7%, 10%, 15%, 20%, 25%, 30%, 40% and 50% by weight.

### Uses

In some embodiments, materials described herein including freeze-dry powder, ground flower powder, phytocannabinoid powder, and materials or compositions comprising such materials, are added to foods and/or beverages for consumption by humans, mammals and other animals, including pets and livestock. Foods and beverages include, but are not limited to, whole foods and beverages, ingredients of foods or beverages including powders or concentrates from which beverages are made, nutritional supplements, pet and livestock feed, pet treats or supplements, and the like.

In some embodiments, the phytocannabinoid powder, freeze-dry powder, liquid from pressing (fresh or frozen) and/or ground flower powder is added to foods and/or beverages, including ingredients in making foods or beverages, to serve as an emulsifier. An emulsifier is generally present in quantities of 0.5% to 50% by weight, including 1%, 5%, 10%, 20%, 25%, 30% and 40% by weight of the food, beverage or ingredient.

### Example 1

A group of twelve Cannabis sativa hemp plants (strain: ACDC) in the eighth week of their flowering cycle were tested for cannabinoid production. Testing began at week 3 of flowering identified as the first sign of resin production by visual inspection by naked eye and 4× magnification. At week eight, 10 lbs (4.5 kg) of biomass was harvested and run through a Vincent press. The collected biomass of flowers and leaves was pressed within 1.5 hours of cutting with a screw press until no further juice production was observed. The liquid portion (about 13.265 Liters) was collected, transferred to a glass vessel and placed in a Labconco FreeZone 6 Liter Benchtop freeze-drying machine at a temperature of −50° C. for 24 hours. The resulting dried solids, approximately 1 lb (450 g), were then made shelf stable and ready for blending. The resulting material contained cannabinoids as shown below

### SUMMARY

| | Total (mg/g) | Percentage |
|---|---|---|
| CBC | 0.53 | 0.05% |
| CBD | 5.79 | 0.58% |
| Δ8-THC | 0.17 | 0.02% |
| Δ9-THC | 0.25 | 0.03% |
| CBG | 0.25 | 0.03% |
| CBN | 0.06 | 0.01% |

Cannabinoid concentration reported as total cannabinoid (Total THC = THCA + THC).
Analyte is below the minimum detection limit (<MDL) of the instrument.

Additionally, 1 lb (450 g) of dried hemp, containing approximately 10% CBD and 0.025% THC was ground to a fine powder (300 mesh) utilizing a commercial grinder. This flower was then prepared for blending and stored.

### Example 2

3 grams of freeze-dry powder and 10 grams of ground flower powder from Example 1 were combined by simple mixing to create 13 grams of phytocannabinoid powder that includes the following cannabinoids (by weight): 0.025% THC, 0.88% CBD, 0.04% CBC, and 0.12% CBN.

### Example 3

To create a phytocannabinoid powder having a custom profile of 0.25% THC, 0.10% CBD, 0.04% CBC, 0.3% CBG and 0.12% CBN of the phytocannabinoid powder from Example 2 was blended utilizing a commercial mixer. Additionally, approximately 12. milligrams of CBD isolate that was isolated in the lab from the original biomass was added to the mix. This isolated CBD was created post pressing via supercritical CO₂ and chromatography.

What is claimed is:

1. A method of producing a customizable phytocannabinoid powder, the method comprising:
harvesting a biomass comprising flowers and leafy materials from a cannabis plant showing signs of resin production;
pressing the biomass to form a liquid content and a solid content;
lyophilizing the liquid content and optionally grinding or sifting the liquid content following lyophilization to form a freeze-dry powder;
drying and then sonicating the solid content to form a ground flower powder; and
mixing at least portion of the ground flower powder with at least a portion of the freeze-dry powder to form a phytocannabinoid powder comprising one or more cannabinoids.

2. The method of claim 1, wherein the cannabis plant is continuously harvested to remove flowers and other leafy materials showing signs of resin production starting from week 3 through week 13 of the growth cycle of the plant.

3. The method of claim 1, further comprising flash-freezing the biomass prior to pressing within 3 hours of harvest at a temperature at or below −30° F.

4. The method of claim 1, further comprising chemically extracting cannabinoids from the solid content following drying.

5. The method of claim 1, wherein the cannabis plant is selected from the group consisting of: Cannabis sativa, Cannabis indica, Cannabis ruderalis, and hybrids of the foregoing.

6. The method of claim 1, wherein the cannabinoids comprise one or more compounds selected from the group consisting of: cannabigerolic acid (CBG-A), cannabigerol (CBG), tetrahydrocannabinolic acid (THC-A), tetrahydrocannabinol-C4 (THC-C4), tetrahydrocannabivarinic acid (THCV-A), delta-9-tetrahydrocannabinol (Δ9THC), delta-8-tetrahydrocannabinol (Δ8THC), tetrahydrocannabivarin (THCV), cannabinolic acid (CBN-A), cannabinol (CBN), cannabidiolic acid (CBD-A), cannabidivaric acid (CBDV-A), cannabidiol (CBD), cannabidivarin (CBDV), cannabichroic acid (CBC-A), cannabichromene (CBC), and cannabicyclol acid (CBC-A).

7. The method of claim 1, further comprising infusing additional cannabinoids into the phytocannabinoid powder to create a powder with a custom cannabinoid profile.

8. A cannabinoid powder comprising dried plant cells and cell components of one or more cannabis plants, cannabinoids, cannabis terpenes, chlorophyll and vitamins made according to the method of claim 1.

9. A method of producing a ground flower powder, the method comprising:
harvesting a biomass comprising flowers and leafy materials from a cannabis plant showing signs of resin production;
pressing the biomass to form a liquid content and a solid content;
optionally extracting isolated cannabinoids from the solid content by CO₂ or ethanol via HPLC or CPC; and
grinding down the solid content to form the ground flower powder.

10. The method of claim 9, wherein the cannabis plant is continuously harvested to remove flowers and other leafy materials showing signs of resin production starting from week 3 through week 13 of the growth cycle of the plant.

11. The method of claim 9, further comprising flash-freezing the biomass prior to pressing within 3 hours of harvest at a temperature at or below −30° F.

12. The method of claim 9, further comprising isolating specific cannabinoids or cannabinoid fractions.

13. The method of claim 9, wherein the cannabis plant is selected from the group consisting of: Cannabis sativa, Cannabis indica, Cannabis ruderalis, and hybrids of the foregoing.

14. The method of claim 9, wherein the cannabinoids comprise one or more compounds selected from the group consisting of: cannabigerolic acid (CBG-A), cannabigerol (CBG), tetrahydrocannabinolic acid (THC-A), tetrahydrocannabinol-C4 (THC-C4), tetrahydrocannabivarinic acid (THCV-A), delta-9-tetrahydrocannabinol (Δ9THC), delta-8-tetrahydrocannabinol (Δ8THC), tetrahydrocannabivarin (THCV), cannabinolic acid (CBN-A), cannabinol (CBN), cannabidiolic acid (CBD-A), cannabidivaric acid (CBDV-A), cannabidiol (CBD), cannabidivarin (CBDV), cannabichroic acid (CBC-A), cannabichromene (CBC), and cannabicyclol acid (CBC-A).

15. The method of claim 9, further comprising infusing additional cannabinoids into the ground flower powder to create a powder with a custom cannabinoid profile.

16. A cannabis plant powder comprising finely ground dried plant cells and cell components of one or more cannabis plants, cannabinoids, and cannabis terpenes, made according to the method of claim 9.

17. A method of making a cannabis juice composition, comprising
harvesting a biomass comprising flowers and leafy materials from a cannabis plant showing signs of resin production;
pressing the biomass to form a liquid content and a solid content;
collecting the liquid content;
optionally lyophilizing the liquid content and further optionally grinding or sifting the liquid content following lyophilization to form a freeze-dry powder.

18. The method of claim 17, wherein the cannabis plant is continuously harvested to remove flowers and other leafy materials showing signs of resin production starting from week 3 through week 13 of the growth cycle of the plant.

19. The method of claim 17, further comprising flash-freezing the biomass prior to pressing within 3 hours of harvest at a temperature at or below −30° F.

20. The method of claim 17, wherein the cannabis plant is selected from the group consisting of: Cannabis sativa, Cannabis indica, Cannabis ruderalis, and hybrids of the foregoing.

21. The method of claim 17, wherein the cannabinoids comprise one or more compounds selected from the group consisting of: cannabigerolic acid (CBG-A), cannabigerol (CBG), tetrahydrocannabinolic acid (THC-A), tetrahydrocannabinol-C4 (THC-C4), tetrahydrocannabivarinic acid (THCV-A), delta-9-tetrahydrocannabinol (Δ9THC), delta-8-tetrahydrocannabinol (Δ8THC), tetrahydrocannabivarin (THCV), cannabinolic acid (CBN-A), cannabinol (CBN), cannabidiolic acid (CBD-A), cannabidivaric acid (CBDV-A), cannabidiol (CBD), cannabidivarin (CBDV), cannabichroic acid (CBC-A), cannabichromene (CBC), and cannabicyclol acid (CBC-A).

22. A cannabinoid powder comprising lyophilized liquid content made according to the method of claim 17, wherein the lyophilized liquid content comprises aqueous and oily components of one or more cannabis plants comprising cannabinoids, cannabis terpenes, chlorophyll and vitamins.

23. A cannabinoid liquid comprising aqueous and oily components of one or more cannabis plants comprising cannabinoids, cannabis terpenes, chlorophyll and vitamins pressed from one or more cannabis plants and made according to the method of claim 17.
